(19) United States
(12) Patent Application Publication
(10) Pub. No.: US 2026/0315753 A1
(43) Pub. Date: Oct. 15, 2026

(54) GUIDEWIRE STABILIZING APPARATUS AND METHODS OF USE AND MANUFACTURE

(71) Applicant: Inari Medical, Inc., Irvine, CA (US)

(72) Inventors: Ben Tsai, Claremont, CA (US); Hamid Rafi, Irvine, CA (US)

(21) Appl. No.: 19/483,941

(22) PCT Filed: May 13, 2024
(86) PCT No.: PCT/US2024/029102

§ 371 Date: Nov. 12, 2025

Related U.S. Application Data
(60) Provisional application No. 63/467,470, filed on May 18, 2023.

(51) Int. Cl. A61M 25/09; A61M 25/02

(52) U.S. Cl. CPC A61M 25/09041; A61M 2025/0266; A61M 2025/028; A61M 2205/583

(57) ABSTRACT

A guidewire stabilizing apparatus is disclosed, with the apparatus including a base; first, second, third and fourth lateral sides, with the first lateral side having a first angled wire guide and the second lateral side having a second angled wire guide; and a fifth, upper side having a stabilization channel extending across the upper side and terminating in the first angled wire guide in the first lateral side and the second angled wire guide in the second lateral side. The first and second lateral sides each further comprise: a first stabilization slit extending from the respective first and second angled wire guides; and a second stabilization slit extending across the first stabilization slit to form a plurality of deformable flaps in each of the respective first and second lateral sides. Methods of fabricating and using the guidewire stabilizing apparatus are also disclosed.

### CROSS-REFERENCE TO A RELATED APPLICATION

This application is a nonprovisional of and claims priority to and the benefit of U.S. Provisional Patent Application No. 63/467,470, filed May 18, 2023, inventors Ben Tsai et al., titled “Guidewire Stabilizing Apparatus and Methods of Use and Manufacture”, and further claims priority to and the benefit of U.S. Design Patent Application No. Ser. No. 29/941,768, filed May 11, 2024, inventors Ben Tsai et al., titled “Guidewire Stabilizing Apparatus”, which are commonly assigned herewith, incorporated herein by reference with the same full force and effect as if set forth in their entireties herein, and with priority claimed for all commonly disclosed subject matter.

### FIELD OF THE INVENTION

The present invention, in general, relates to the medical and surgical catheterization procedures, and more particularly, relates to an apparatus for stabilizing a guidewire utilized in such catheterization procedures and methods for use and manufacturing of the apparatus.

### BACKGROUND OF THE INVENTION

Catheterization procedures may be performed for treatment of a wide variety of medical problems and issues in human and non-human patients or subjects, such as for removal of blood clots and emboli from veins or arteries, during transcatheter heart valve procedures, or during ultrasound imaging procedures, for example and without limitation. A guidewire is typically inserted into the subject vein or artery and advanced within the vein or artery to the selected or desired location within the patient or subject, followed by inserting and advancing a clot removal catheter or other type of catheter to that selected or desired location. The guidewire aids in properly orienting the catheter throughout the medical, surgical or other interventional catheterization procedure.

As the catheter is being utilized and moved during the procedure, the guidewire also has a tendency to move or “walk”. In the event the guidewire were to move too far from the selected or desired location, the procedure may have to be interrupted to correct for this movement and move the guidewire back to the selected or desired location. To avoid this problem during the medical, surgical or other interventional catheterization procedure, additional support staff or other personnel are typically employed to stabilize the guidewire, such as by using a finger, thumb, or hand to hold the guidewire against the operating or procedure table. Such additional staff may not always be available. Potential solutions such as taping the guidewire down to the operating or procedure table have also been shown to be inadequate. In addition, if the guidewire is not stabilized properly, it can slip or move excessively, such as out of visualization range on the monitor used in the procedure. All of these issues can result in added personnel expense and delay of the medical, surgical or other interventional catheterization procedure.

Accordingly, a need remains for an apparatus specifically designed for the stabilizing of a guidewire during a medical, surgical or other interventional catheterization procedure. Once placed and holding the guidewire, such an apparatus should be capable of being utilized without ongoing involvement of additional personnel. Such an apparatus should be able to be placed stably and securely on the operating or procedure table, and should securely hold and stabilize the guidewire throughout the entirety of the medical, surgical or other interventional catheterization procedure. Such an apparatus should also be readily and easily deployable by medical personnel, without significant added training in its utilization. Such an apparatus should be capable of being integrated and included into the overall, sterile packaging of the catheterization instruments, to be provided as part of a single, complete set or kit of instruments for the medical, surgical or other interventional catheterization procedure. In addition, such an apparatus should be readily manufacturable and comparatively low cost to produce and deploy.

### SUMMARY OF THE INVENTION

The exemplary or representative embodiments of the present invention provide numerous advantages. Various representative embodiments provide an apparatus specifically designed for the stabilizing of a guidewire during a medical, surgical or other interventional catheterization procedure. Once placed and holding the guidewire, the representative apparatus can be utilized without ongoing involvement of additional personnel. The representative apparatus can be placed stably and securely on the operating or procedure table, and securely holds and stabilizes the guidewire throughout the entirety of the medical, surgical or other interventional catheterization procedure. The representative apparatus is readily and easily deployable by medical personnel, without significant added training in its utilization. The representative apparatus is capable of being integrated and included into the overall, sterile packaging of the catheterization instruments, to be provided as part of a single, complete set or kit of instruments for the medical, surgical or other interventional catheterization procedure. In addition, the representative apparatus is readily manufacturable, by cutting and folding, and comparatively low cost to produce and deploy.

In a representative embodiment, a guidewire stabilizing apparatus comprises: a base; a plurality of lateral sides coupled to or integrally formed with the base, the plurality of lateral sides comprising a first lateral side, a second lateral side opposite the first lateral side, a third lateral side, and a fourth lateral side opposite the third lateral side, the first lateral side comprising a first angled wire guide and the second lateral side comprising a second angled wire guide; and a fifth, upper side coupled to or integrally formed with the first, second, third, and fourth lateral sides, the fifth, upper side comprising: a stabilization channel extending across the fifth, upper side and terminating in the first angled wire guide in the first lateral side and in the second angled wire guide in the second lateral side; wherein the first and second lateral sides each further comprise: a first stabilization slit extending toward the base from the respective first and second angled wire guides; and a second stabilization slit extending across the first stabilization slit to form a plurality of deformable flaps in each of the respective first and second lateral sides. In a representative embodiment, the second stabilization slit may be arranged at a right angle to the first stabilization slit.

In a representative embodiment, the base may further comprise a plurality of through-holes. In a representative embodiment, the first lateral side may further comprise: a first tab; and a second tab; wherein the first and second tabs each extend through a first through-hole, of the plurality of through-holes of the base, the first and second tabs further arranged along a first, lower surface of the base. In a representative embodiment, the first lateral side may further comprise: an extension panel arranged on a second, upper surface of the base and extending between the fifth, upper side and the second, upper surface of the base.

In such a representative embodiment, the third lateral side may further comprise: a third tab extending through a second through-hole, of the plurality of through-holes of the base, the third tab further arranged along the first, lower surface of the base. Also in such a representative embodiment, the fourth lateral side may further comprise: a fourth tab extending through a third through-hole, of the plurality of through-holes of the base, the fourth tab further arranged along the first, lower surface of the base.

In a representative embodiment, the guidewire stabilizing apparatus may further comprise: an adhesive layer having a first adhesive side and a second adhesive side, the first adhesive side of the adhesive layer coupled to a first, lower surface of the base; and a removable adhesive cover sheet coupled to the second adhesive side of the adhesive layer.

In a representative embodiment, each of the first angled wire guide and the second angled wire guide may be V-shaped.

A representative embodiment of a method of using the guidewire stabilizing apparatus is also disclosed, with the method comprising: removing the removable adhesive cover sheet; positioning and pressing the second adhesive side of the adhesive layer to a sterile covering of an operating or procedure table; and inserting a guidewire into the stabilization channel, into the first angled wire guide and the first stabilization slit of the first lateral side, and into the second angled wire guide and first stabilization slit of the second lateral side.

A representative embodiment of a method of manufacturing or fabricating the guidewire stabilizing apparatus from a sheet of a substrate material having a plurality of through-cuts apparatus is also disclosed, with the plurality of through-cuts forming the first, second, third, and fourth tabs and the first, second, and third through-holes, and with the sheet of a substrate material further having a plurality of fold lines. A representative method comprises: folding the sheet of a substrate material along the plurality of fold lines; inserting the first and second tabs into the first through-hole; inserting the third tab into the second through-hole; and inserting the fourth tab into the third through-hole. In a representative embodiment, an adhesive layer comprises a first adhesive side, a second adhesive side, and a removable adhesive cover sheet on the second adhesive side, with the representative method further comprising: coupling the first adhesive side of the adhesive layer to the first, lower surface of the base.

In a representative embodiment, the first and second tabs may be integrally formed with the first lateral side, the third tab may be integrally formed with the third lateral side, and the fourth tab may be integrally formed with the fourth lateral side.

In a representative embodiment, the base may further comprise: one or more indicia or indicators, the one or more indicia or indicators configured to indicate at least one indication selected from the group consisting of: an orientation of the guidewire stabilizing apparatus with respect to a catheter access point, a removal from packaging, a location for insertion of a guidewire into the stabilization channel, the first or second angled wire guides, and one or more combinations thereof.

Also in a representative embodiment, the guidewire stabilizing apparatus may comprises a material selected from the group consisting of: polyethylene, polycarbonate, polypropylene, polyethylene terephthalate, or high-density polyethylene having a molecular weight between 100,000-250,000 Daltons.

In a representative embodiment, the first stabilization slit and the second stabilization slit may each have a length between 1/16^{th} to 3/16^{th} of an inch (1.5875 to 4.7625 mm). In such a representative embodiment, each of the base, the first lateral side, the second lateral side, the third lateral side, the fourth lateral side, and the fifth, upper side may each have a thickness ranging from 0.020 to 0.035 inches (0.508 to 0.889 mm).

In such a representative embodiment, the first stabilization slit and second stabilization slit are configured to secure a guidewire having a diameter at or between 0.014 inch (0.3556 mm) to 0.035 inch (0.889 mm) against an applied force of 0.50 lbf (2.224 N).

In a representative embodiment, the guidewire stabilizing apparatus is foldable from a single sheet of substrate material. In a representative embodiment, the third lateral side and the fourth lateral side are each coupled between the first and second lateral sides.

In a representative embodiment, the guidewire stabilizing apparatus may further comprise or be included in a kit of catheterization instruments, with the kit of catheterization instruments also including or comprising: a catheter; a syringe; and a packaging tray having a plurality of tabs or other packaging tray extensions configured to hold or secure each of the guidewire stabilizing apparatus, the catheter, and the syringe.

In another representative embodiment, a guidewire stabilizing apparatus may comprise: a base, the base having a plurality of through-holes; a plurality of lateral sides coupled to or integrally formed with the base, the plurality of lateral sides comprising a first lateral side, a second lateral side opposite the first lateral side, a third lateral side, and a fourth lateral side opposite the third lateral side, the first lateral side comprising a first angled wire guide and the second lateral side comprising a second angled wire guide; a fifth, upper side coupled to or integrally formed with the first, second, third, and fourth lateral sides, the fifth, upper side comprising: a stabilization channel extending across the fifth, upper side and terminating in the first angled wire guide in the first lateral side and in the second angled wire guide in the second lateral side; an adhesive layer having a first adhesive side and a second adhesive side, the first adhesive side of the adhesive layer coupled to a first, lower surface of the base; and a removable adhesive cover sheet coupled to the second adhesive side of the adhesive layer; wherein the first and second lateral sides each further comprise: a first stabilization slit extending toward the base from the respective first and second angled wire guides; and a second stabilization slit extending across the first stabilization slit to form a plurality of deformable flaps in each of the respective first and second lateral sides.

In another representative embodiment, a guidewire stabilizing apparatus may comprise: a base; a plurality of lateral sides coupled to or integrally formed with the base, a first lateral side of the plurality of lateral sides comprising a first angled wire guide and a second lateral side of the plurality of lateral sides comprising a second angled wire guide; and an upper side coupled to or integrally formed with the plurality of lateral sides, the upper side comprising: a stabilization channel extending across the upper side and terminating in the first angled wire guide in the first lateral side and in the second angled wire guide in the second lateral side; wherein the first and second lateral sides each further comprise: a first stabilization slit extending toward the base from the respective first and second angled wire guides; and a second stabilization slit extending across the first stabilization slit and at a right angle to the first stabilization slit to form a plurality of deformable flaps in each of the respective first and second lateral sides.

In another representative embodiment, a guidewire stabilizing apparatus may comprise: a base, the base having a plurality of through-holes; a plurality of lateral sides coupled to or integrally formed with the base, the plurality of lateral sides comprising a first lateral side, a second lateral side opposite the first lateral side, a third lateral side, and a fourth lateral side opposite the third lateral side, the first lateral side comprising a first angled wire guide and the second lateral side comprising a second angled wire guide, and the first, third, and fourth lateral sides each having one or more tabs coupled in a corresponding through-hole of the plurality of through-holes of the base; a fifth, upper side coupled to or integrally formed with the first, second, third, and fourth lateral sides, the fifth, upper side comprising: a stabilization channel extending across the fifth, upper side and terminating in the first angled wire guide in the first lateral side and in the second angled wire guide in the second lateral side; an adhesive layer having a first adhesive side and a second adhesive side, the first adhesive side of the adhesive layer coupled to a lower surface of the base; and a removable adhesive cover sheet coupled to the second adhesive side of the adhesive layer; wherein the first and second lateral sides each further comprise: a first stabilization slit extending toward the base from the respective first and second angled wire guides; and a second stabilization slit extending across the first stabilization slit and at a right angle to the first stabilization slit to form a plurality of deformable flaps in each of the respective first and second lateral sides.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1A is an isometric view illustrating a representative first embodiment of a guidewire stabilizing apparatus, in a folded configuration.

FIG. 1B is an enlarged, side elevational view of a portion of FIG. 1A illustrating individual stabilization slits (of a first pair of stabilization slits) of the representative first embodiment of the guidewire stabilizing apparatus, in the folded configuration.

FIGS. 2A and 2B (collectively referred to as “FIG. 2”) are plan views illustrating the representative first embodiment of the guidewire stabilizing apparatus, in an unfolded, flat configuration.

FIG. 3 is a top, plan view illustrating the representative first embodiment of the guidewire stabilizing apparatus, in the folded configuration.

FIG. 4 is a first side elevational view illustrating the representative first embodiment of the guidewire stabilizing apparatus, in the folded configuration, without an adhesive layer and an adhesive cover sheet.

FIG. 5 is a second side elevational view illustrating the representative first embodiment of the guidewire stabilizing apparatus, in the folded configuration, without the adhesive layer and the adhesive cover sheet.

FIG. 6 is a third side elevational view illustrating the representative first embodiment of the guidewire stabilizing apparatus, in the folded configuration, without the adhesive layer and the adhesive cover sheet.

FIG. 7 is a fourth side elevational view illustrating the representative first embodiment of the guidewire stabilizing apparatus, in the folded configuration, without the adhesive layer and the adhesive cover sheet.

FIG. 8 is a bottom, plan view illustrating the representative first embodiment of the guidewire stabilizing apparatus, in the folded configuration, without the adhesive layer and the adhesive cover sheet.

FIG. 9 is a bottom, plan view illustrating the representative first embodiment of the guidewire stabilizing apparatus, in the folded configuration, with the adhesive layer and the adhesive cover sheet.

FIG. 10 is an isometric view illustrating the representative first embodiment of the guidewire stabilizing apparatus, in the folded configuration, with a guidewire arranged in the first and second pairs of stabilization slits.

FIG. 11 is a first, enlarged, isometric view of a portion of FIG. 10 illustrating a first arrangement for the guidewire to be arranged in the individual stabilization slits (of the first pair of stabilization slits) of the representative first embodiment of the guidewire stabilizing apparatus, in the folded configuration.

FIG. 12 is a second, enlarged, isometric view of a portion of FIG. 10 illustrating a second arrangement for the guidewire to be arranged in the individual stabilization slits (of the first pair of stabilization slits) of the representative first embodiment of the guidewire stabilizing apparatus, in the folded configuration.

FIG. 13 is a plan view illustrating the representative first embodiment of the guidewire stabilizing apparatus, in the folded configuration, with the guidewire arranged in the first and second pairs of stabilization slits.

FIG. 14 is a side, elevational view illustrating the representative first embodiment of the guidewire stabilizing apparatus, in the folded configuration, with the guidewire arranged in the individual stabilization slits (of the first pair of stabilization slits), in the first arrangement, and with the adhesive layer and the adhesive cover sheet.

FIG. 15 is an isometric view illustrating a representative second embodiment of a guidewire stabilizing apparatus.

FIG. 16 is a top, plan view illustrating the representative second embodiment of the guidewire stabilizing apparatus.

FIG. 17 is a first, side elevational view illustrating the representative second embodiment of the guidewire stabilizing apparatus, with the adhesive layer and the adhesive cover sheet.

FIG. 18 is a second, side elevational view illustrating the representative second embodiment of the guidewire stabilizing apparatus, with the adhesive layer and the adhesive cover sheet.

FIG. 19 is a third, side elevational view illustrating the representative second embodiment of the guidewire stabilizing apparatus, with the adhesive layer and the adhesive cover sheet.

FIG. 20 is a fourth, side elevational view illustrating the representative second embodiment of the guidewire stabilizing apparatus, with the adhesive layer and the adhesive cover sheet.

FIG. 21 is a bottom, plan view illustrating the representative second embodiment of the guidewire stabilizing apparatus, with the adhesive layer and the adhesive cover sheet.

FIG. 22 is an isometric view illustrating a representative third embodiment of a guidewire stabilizing apparatus.

FIG. 23 is a top, plan view illustrating the representative third embodiment of the guidewire stabilizing apparatus.

FIG. 24 is a first, side elevational view illustrating the representative third embodiment of the guidewire stabilizing apparatus, with the adhesive layer and the adhesive cover sheet.

FIG. 25 is a second, side elevational view illustrating the representative third embodiment of the guidewire stabilizing apparatus, with the adhesive layer and the adhesive cover sheet.

FIG. 26 is a third, side elevational view illustrating the representative third embodiment of the guidewire stabilizing apparatus, with the adhesive layer and the adhesive cover sheet.

FIG. 27 is a fourth, side elevational view illustrating the representative third embodiment of the guidewire stabilizing apparatus, with the adhesive layer and the adhesive cover sheet.

FIG. 28 is a bottom, plan view illustrating the representative third embodiment of the guidewire stabilizing apparatus, with the adhesive layer and the adhesive cover sheet.

FIG. 29 is a first isometric view illustrating a representative fourth embodiment of a guidewire stabilizing apparatus, in a folded configuration.

FIG. 30 is a second isometric view illustrating the representative fourth embodiment of a guidewire stabilizing apparatus, in the folded configuration.

FIG. 31 is a plan view illustrating the representative fourth embodiment of the guidewire stabilizing apparatus, in an unfolded, flat configuration.

FIG. 32 is a bottom, plan view illustrating the representative fourth embodiment of the guidewire stabilizing apparatus, in the folded configuration, with an adhesive layer and an adhesive cover sheet.

FIG. 33 is an isometric view illustrating a representative embodiment of a kit or set of catheterization instruments having the representative first embodiment of a guidewire stabilizing apparatus, in the folded configuration.

FIG. 34 is a flow chart illustrating a representative embodiment for a method to manufacture the representative first embodiment of the guidewire stabilizing apparatus.

FIG. 35 is a flow chart illustrating a representative embodiment for a method to use the various embodiments of the guidewire stabilizing apparatus in a catheterization procedure.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

As mentioned above, various representative embodiments provide guidewire stabilizing apparatus 100, 200, 300, 400 specifically designed for the stabilizing of a guidewire 90 during a medical, surgical or other interventional catheterization procedure. Once placed and holding the guidewire 90, the representative embodiments of the guidewire stabilizing apparatus 100, 200, 300, 400 can be utilized without ongoing involvement of additional personnel. The representative embodiments of the guidewire stabilizing apparatus 100, 200, 300, 400 can be placed stably and securely on the operating or procedure table, and securely holds and stabilizes the guidewire 90 throughout the entirety of the medical, surgical or other interventional catheterization procedure. The representative embodiments of the guidewire stabilizing apparatus 100, 200, 300, 400 are readily and easily deployable by medical personnel, without significant added training in its utilization. The representative embodiments of the guidewire stabilizing apparatus 100, 200, 300, 400 are capable of being integrated and included into the overall, sterile packaging of the catheterization instruments, to be provided as part of a single, complete kit or set of instruments 500 for the medical, surgical or other interventional catheterization procedure. In addition, the representative embodiments of the guidewire stabilizing apparatus 100, 200, 300, 400 are readily manufacturable and comparatively low cost to produce and deploy.

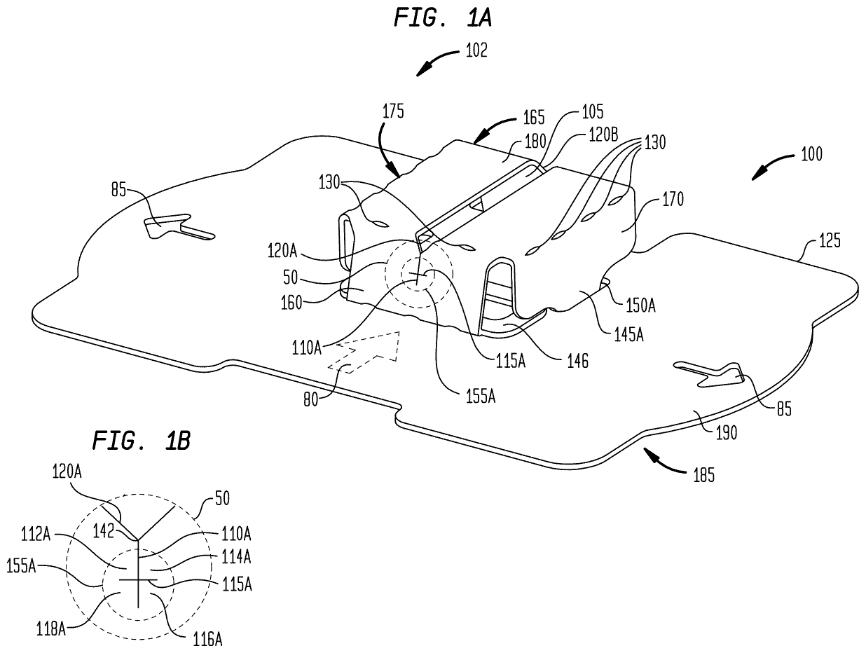

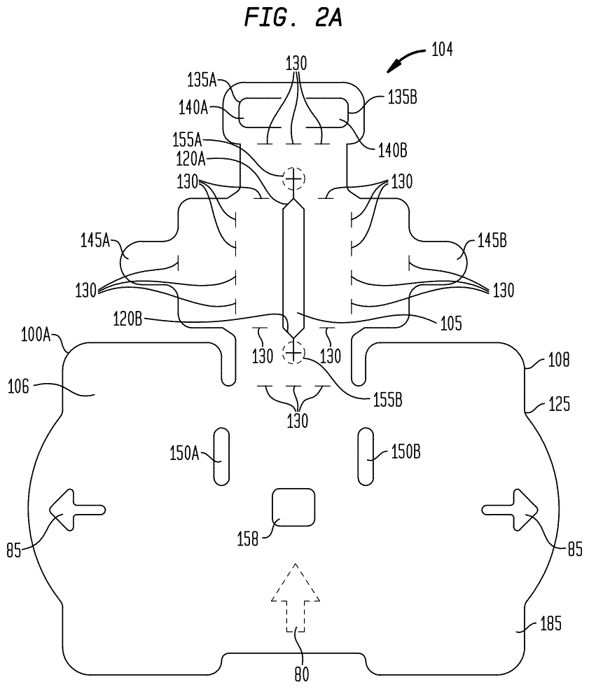

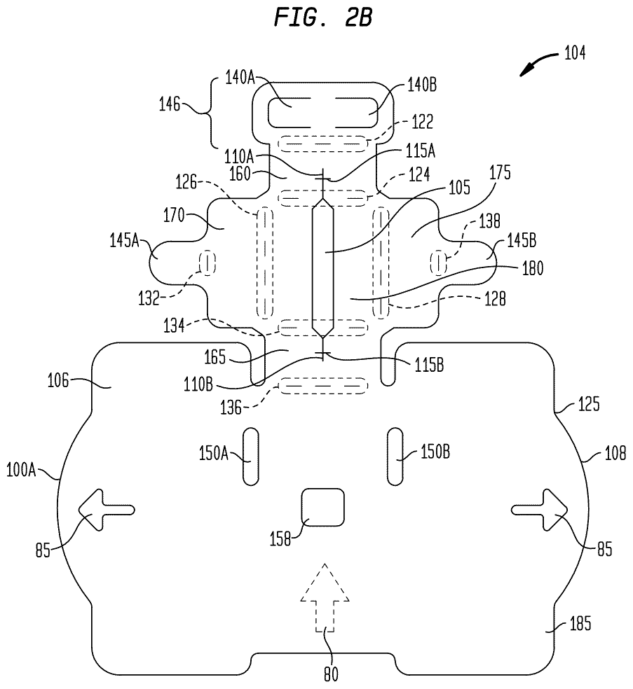

FIG. 1 is an isometric view illustrating a representative first embodiment of a guidewire stabilizing apparatus 100, in a folded configuration 102. FIG. 1B is an enlarged, side elevational view of a portion (50) of FIG. 1A illustrating individual stabilization slits 110A and 115A (of a first pair of stabilization slits 155A) of the representative first embodiment of the guidewire stabilizing apparatus 100, in the folded configuration. FIGS. 2A and 2B are plan views illustrating the representative first embodiment of the guidewire stabilizing apparatus 100A, in an unfolded, flat configuration 104, and are illustrated as separate Figures to better visualize the folding and corresponding reference numerals. It should also be noted that for reasons of visual clarity, not all applicable reference numerals are included in any given Figure.

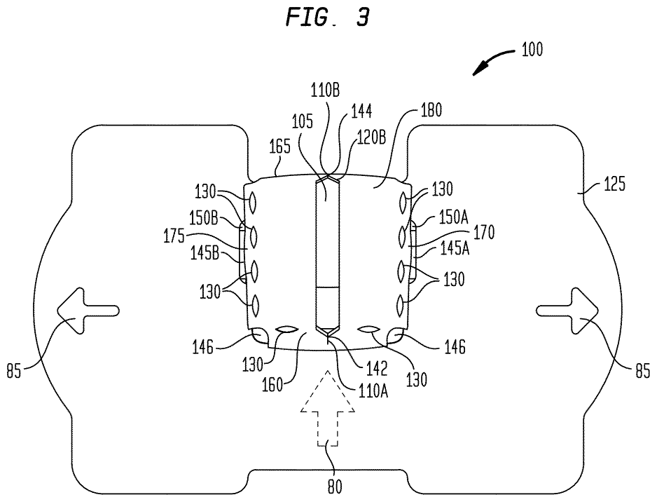

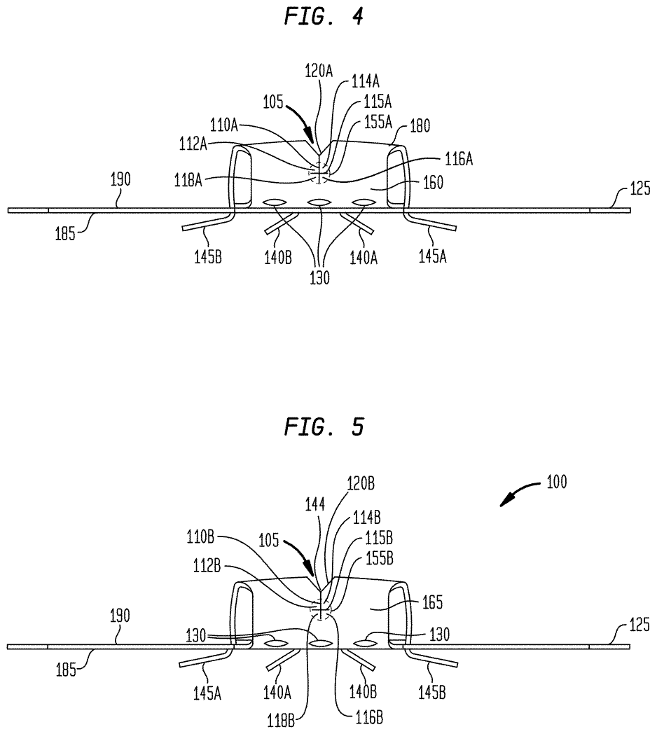

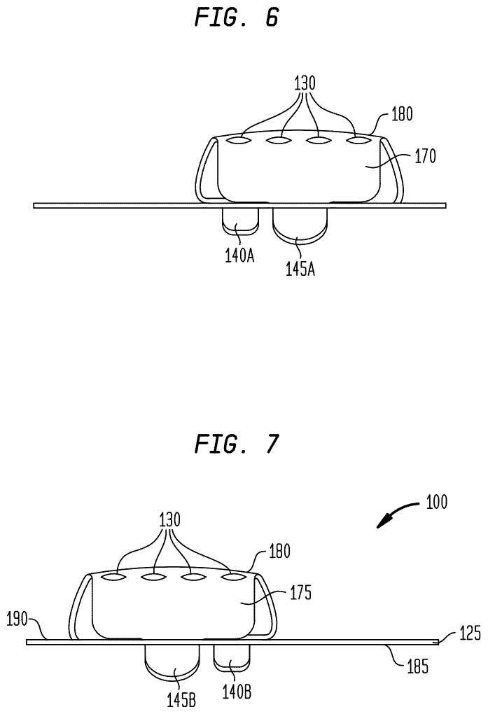

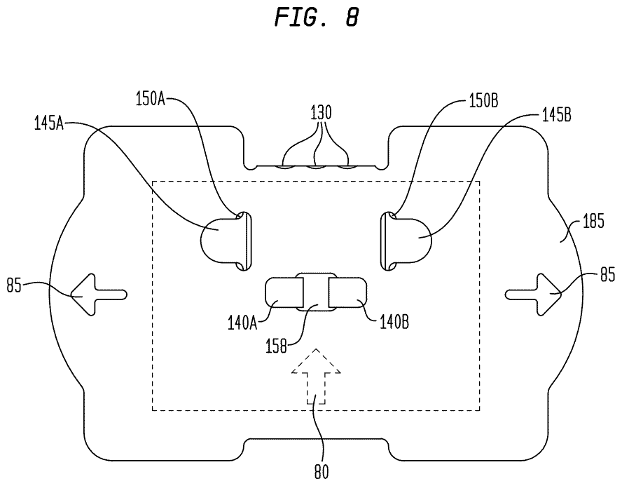

FIG. 3 is a top, plan view illustrating the representative first embodiment of the guidewire stabilizing apparatus 100, in the folded configuration 102. FIG. 4 is a first side elevational view illustrating the representative first embodiment of the guidewire stabilizing apparatus 100, in the folded configuration 102, without an adhesive layer 195 and an adhesive cover sheet 198. FIG. 5 is a second side elevational view illustrating the representative first embodiment of the guidewire stabilizing apparatus 100, in the folded configuration 102, without the adhesive layer 195 and the adhesive cover sheet 198. FIG. 6 is a third side elevational view illustrating the representative first embodiment of the guidewire stabilizing apparatus 100, in the folded configuration 102, without the adhesive layer 195 and the adhesive cover sheet 198. FIG. 7 is a fourth side elevational view illustrating the representative first embodiment of the guidewire stabilizing apparatus 100, in the folded configuration 102, without the adhesive layer 195 and the adhesive cover sheet 198.

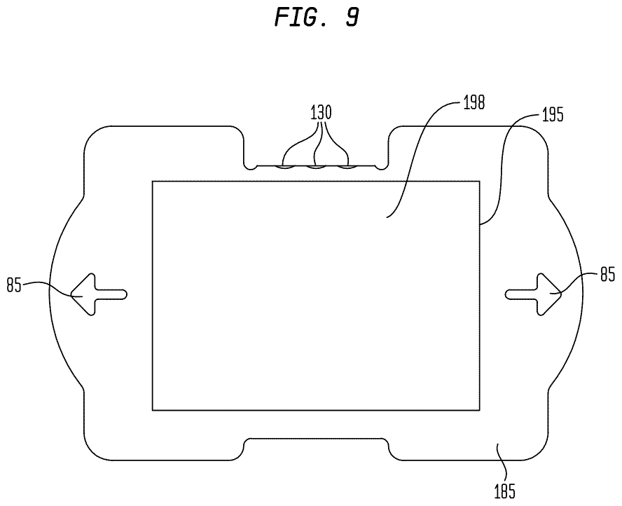

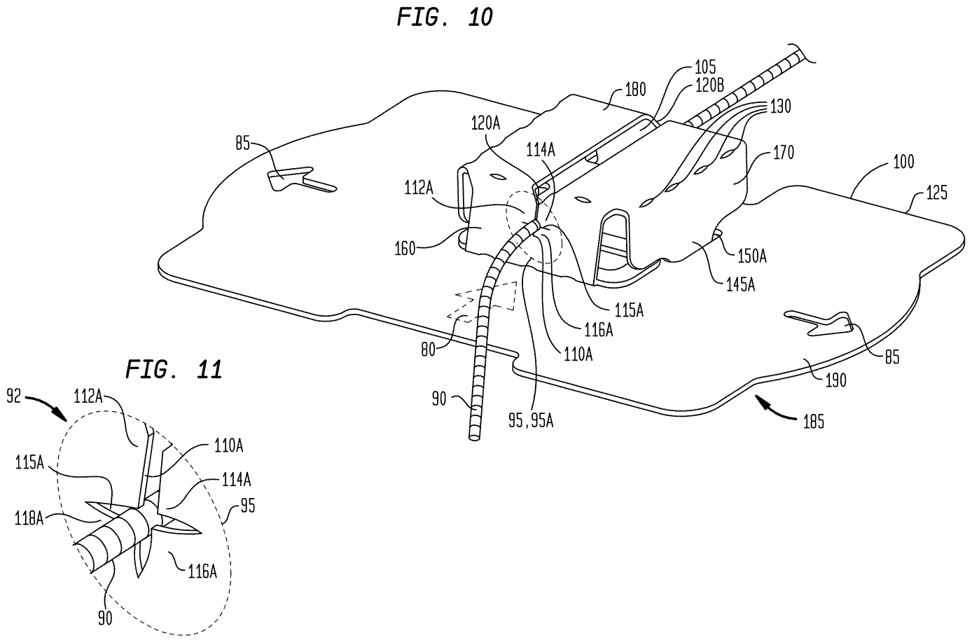

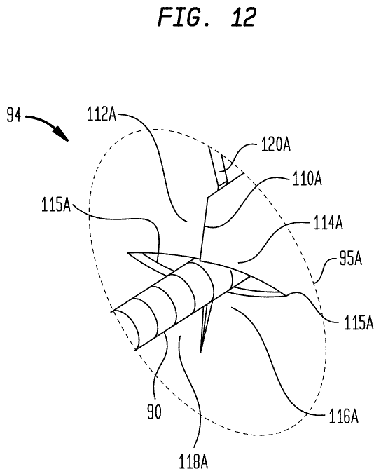

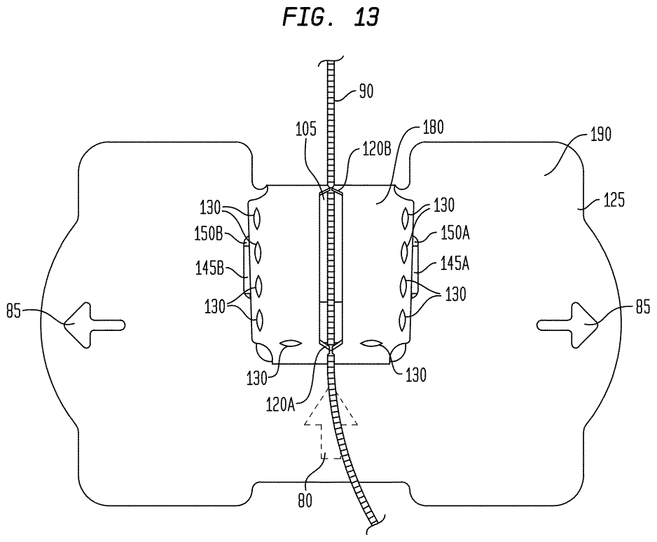

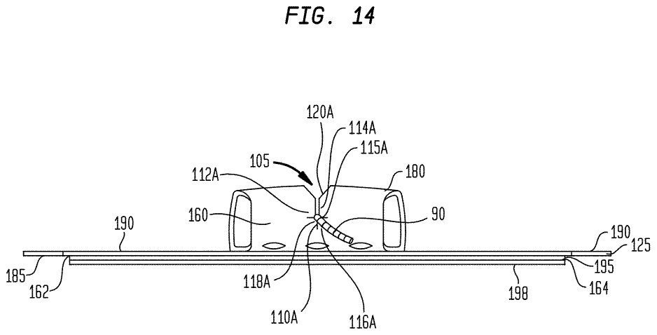

FIG. 8 is a bottom, plan view illustrating the representative first embodiment of the guidewire stabilizing apparatus 100, in the folded configuration 102, without the adhesive layer 195 and the adhesive cover sheet 198. FIG. 9 is a bottom, plan view illustrating the representative first embodiment of the guidewire stabilizing apparatus 100, in the folded configuration 102, with the adhesive layer 195 and the adhesive cover sheet 198. FIG. 10 is an isometric view illustrating the representative first embodiment of the guidewire stabilizing apparatus 100, in the folded configuration 102, with a guidewire 90 arranged in first and second pairs of stabilization slits 155A, 155B. FIG. 11 is a first, enlarged, isometric view of a portion (95) of FIG. 10 illustrating a first arrangement 92 for the guidewire 90 to be arranged in the individual stabilization slits 110A, 115A (of the first pair of stabilization slits 155A) of the representative first embodiment of the guidewire stabilizing apparatus 100, in the folded configuration 102. FIG. 12 is a second, enlarged, isometric view of a portion (95A) of FIG. 10 illustrating a second arrangement 94 for the guidewire 90 to be arranged in the individual stabilization slits 110A, 115A (of the first pair of stabilization slits 155A) of the representative first embodiment of the guidewire stabilizing apparatus 100, in the folded configuration 102. FIG. 13 is a plan view illustrating the representative first embodiment of the guidewire stabilizing apparatus 100, in the folded configuration 102, with the guidewire 90 arranged in the first and second pairs of stabilization slits 155A, 155B. FIG. 14 is a side, elevational view illustrating the representative first embodiment of the guidewire stabilizing apparatus 100, in the folded configuration 102, with the guidewire 90 arranged in the first pair of stabilization slits 155A, and the adhesive layer 195 and the adhesive cover sheet 198.

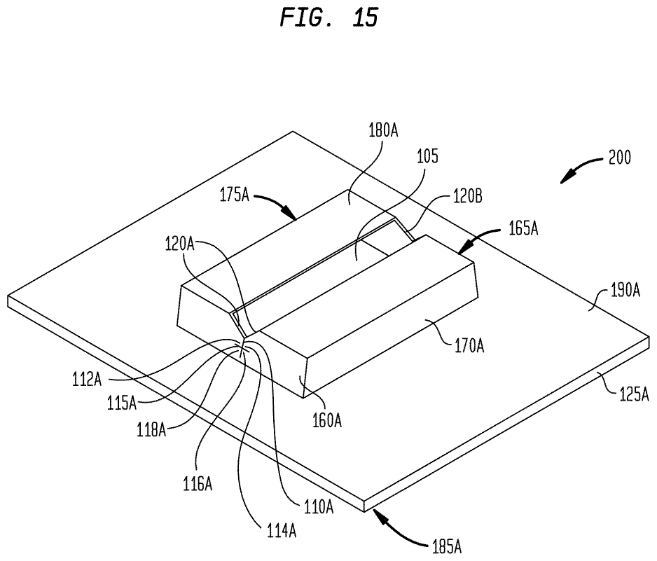

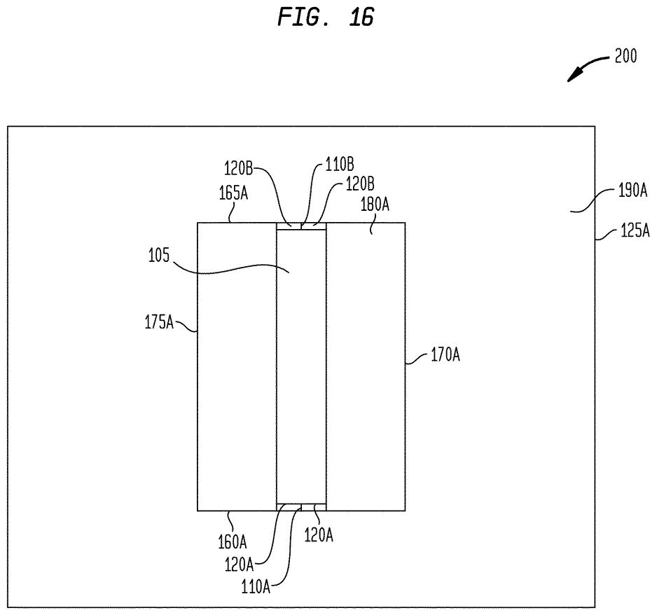

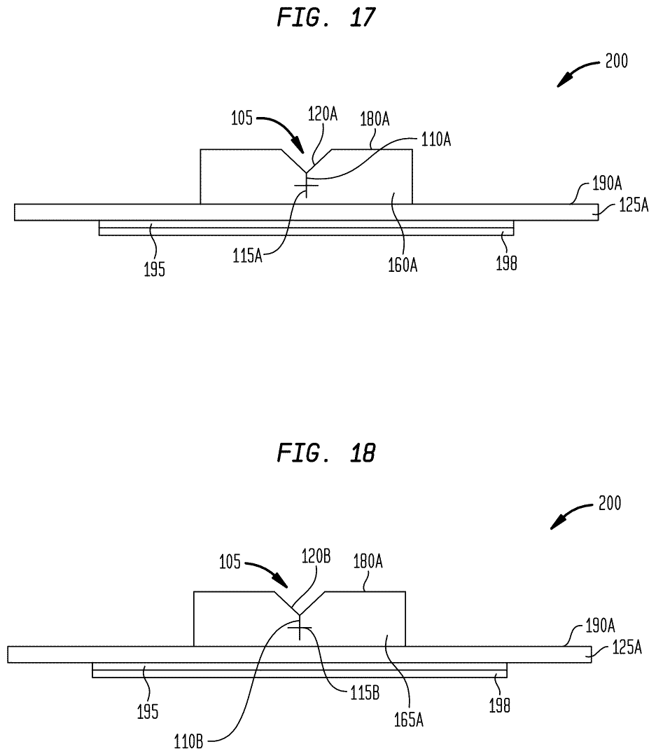

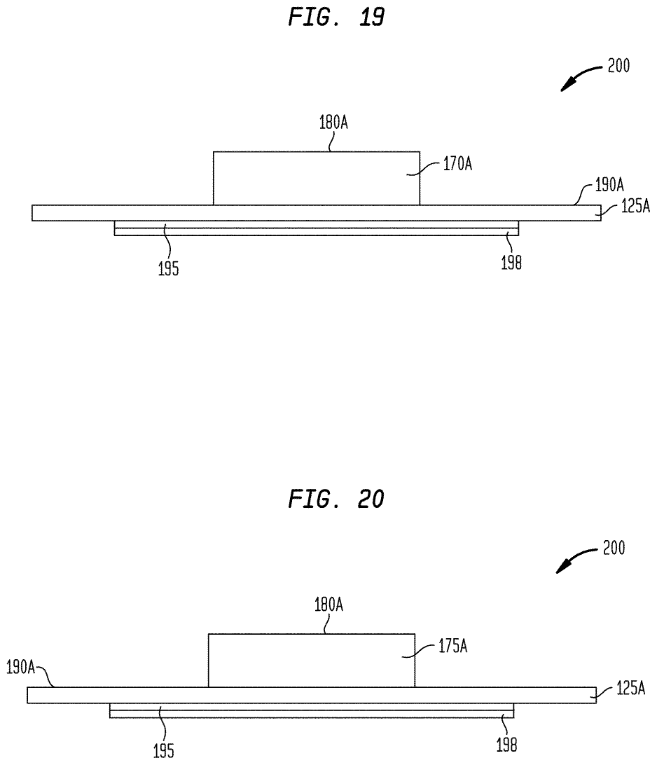

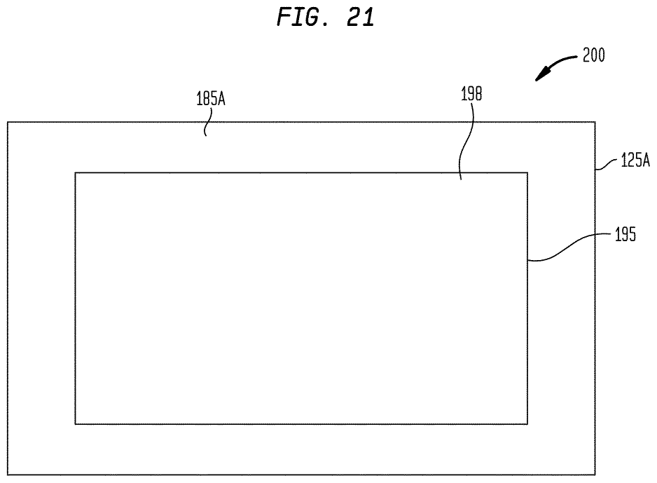

FIG. 15 is an isometric view illustrating a representative second embodiment of a guidewire stabilizing apparatus 200. FIG. 16 is a top, plan view illustrating the representative second embodiment of the guidewire stabilizing apparatus 200. FIG. 17 is a first, side elevational view illustrating the representative second embodiment of the guidewire stabilizing apparatus 200, with the adhesive layer 195 and the adhesive cover sheet 198. FIG. 18 is a second, side elevational view illustrating the representative second embodiment of the guidewire stabilizing apparatus 200, with the adhesive layer 195 and the adhesive cover sheet 198. FIG. 19 is a third, side elevational view illustrating the representative second embodiment of the guidewire stabilizing apparatus 200, with the adhesive layer 195 and the adhesive cover sheet 198. FIG. 20 is a fourth, side elevational view illustrating the representative second embodiment of the guidewire stabilizing apparatus 200, with the adhesive layer 195 and the adhesive cover sheet 198. FIG. 21 is a bottom, plan view illustrating the representative second embodiment of the guidewire stabilizing apparatus 200, with the adhesive layer 195 and the adhesive cover sheet 198.

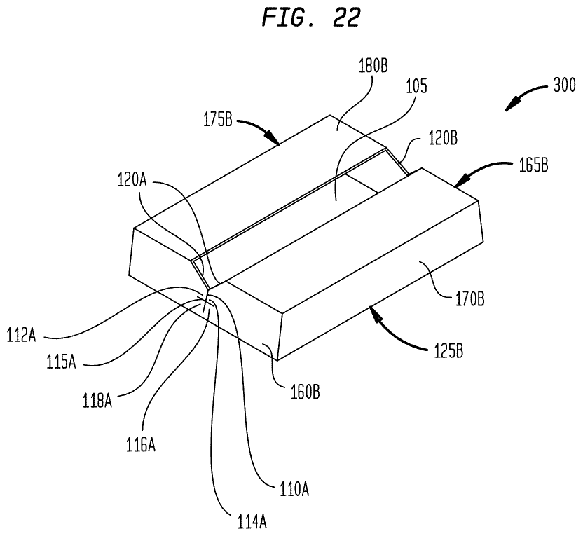

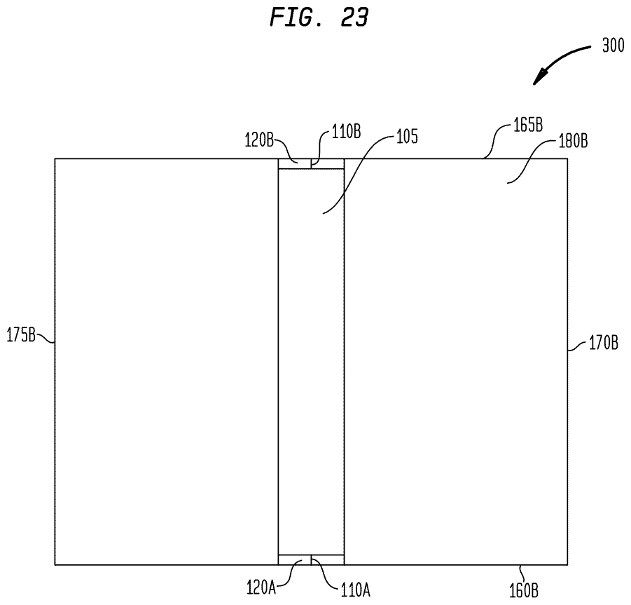

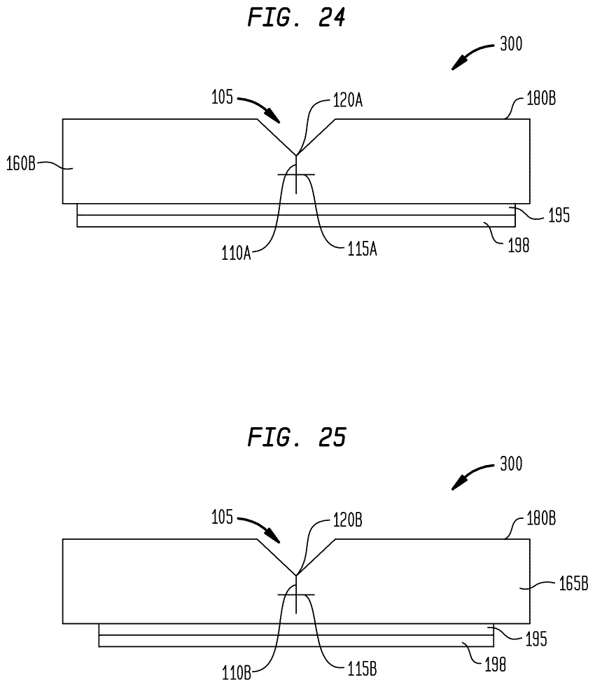

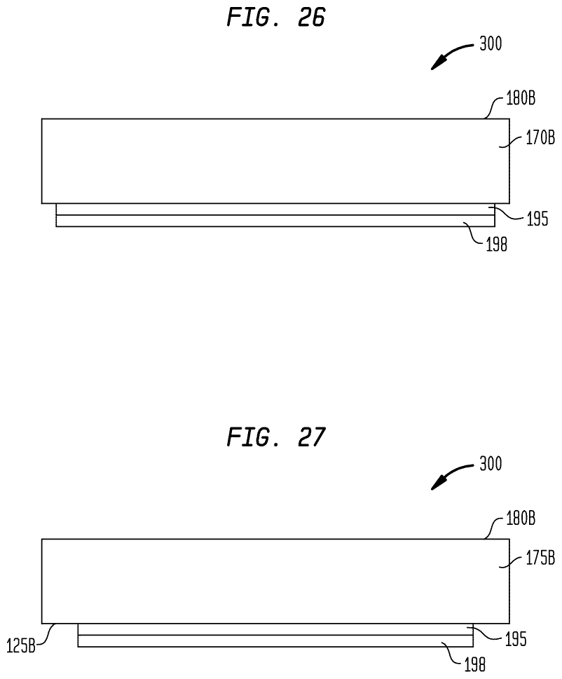

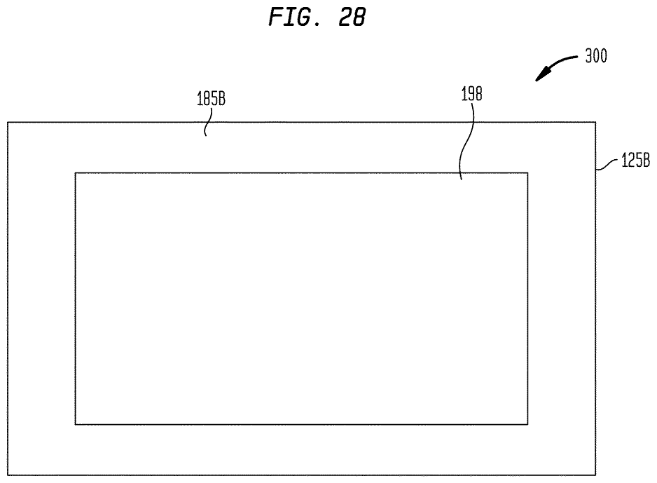

FIG. 22 is an isometric view illustrating a representative third embodiment of a guidewire stabilizing apparatus 300. FIG. 23 is a top, plan view illustrating the representative third embodiment of the guidewire stabilizing apparatus 300. FIG. 24 is a first, side elevational view illustrating the representative third embodiment of the guidewire stabilizing apparatus 300, with the adhesive layer 195 and the adhesive cover sheet 198. FIG. 25 is a second, side elevational view illustrating the representative third embodiment of the guidewire stabilizing apparatus 300, with the adhesive layer 195 and the adhesive cover sheet 198. FIG. 26 is a third, side elevational view illustrating the representative third embodiment of the guidewire stabilizing apparatus 300, with the adhesive layer 195 and the adhesive cover sheet 198. FIG. 27 is a fourth, side elevational view illustrating the representative third embodiment of the guidewire stabilizing apparatus 300, with the adhesive layer 195 and the adhesive cover sheet 198. FIG. 28 is a bottom, plan view illustrating the representative third embodiment of the guidewire stabilizing apparatus 300, with the adhesive layer 195 and the adhesive cover sheet 198.

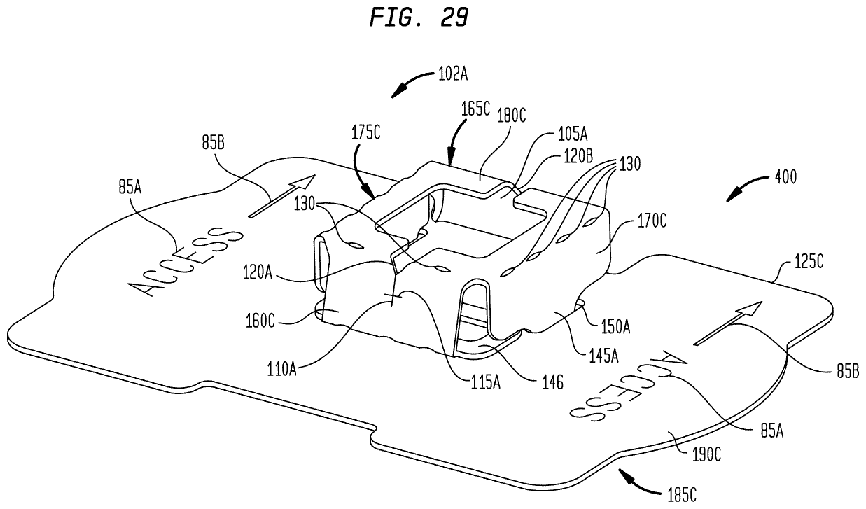

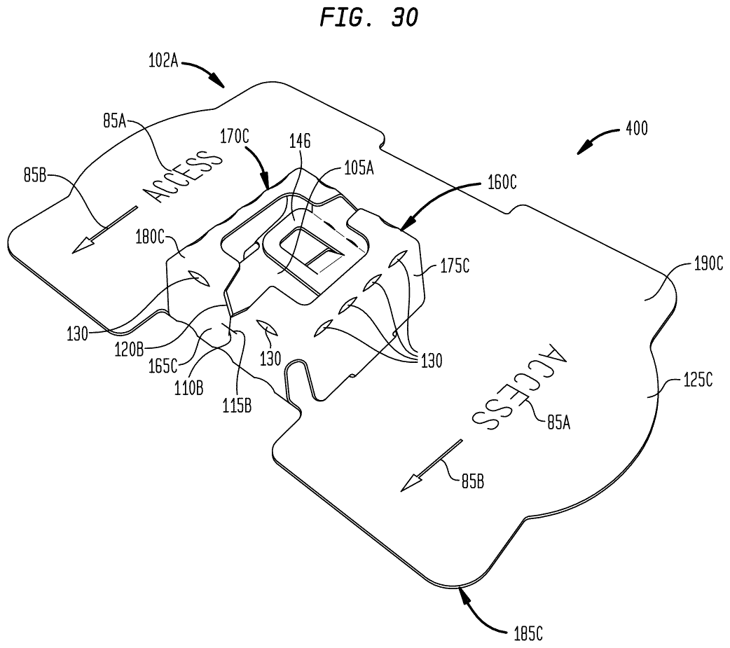

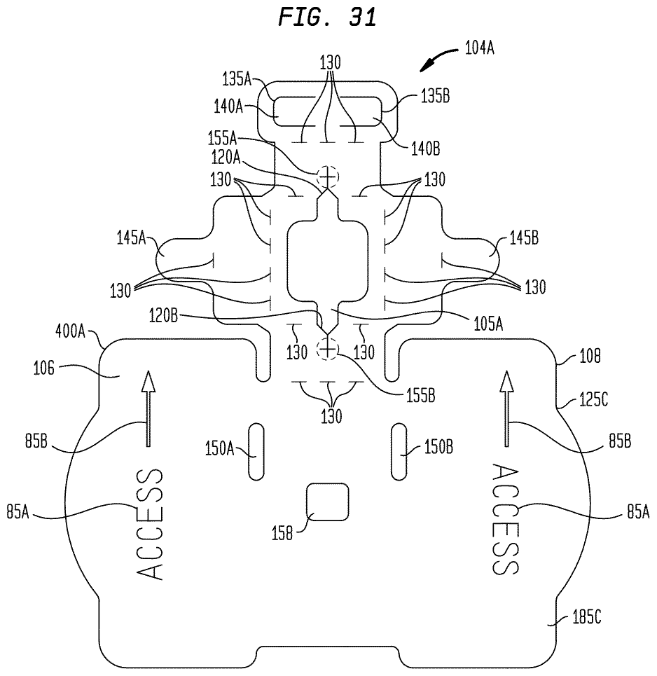

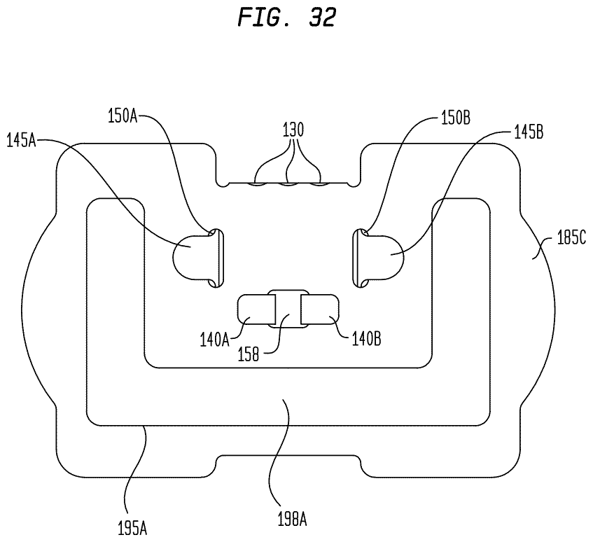

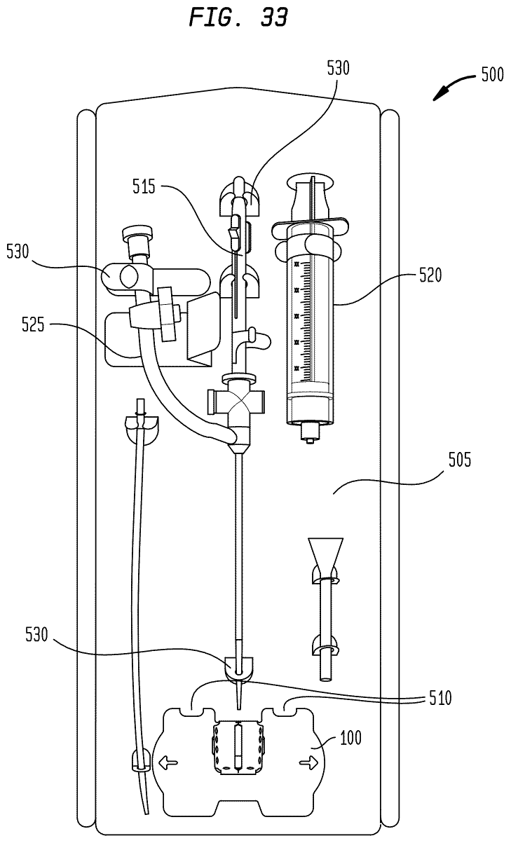

FIG. 29 is a first isometric view illustrating a representative fourth embodiment of a guidewire stabilizing apparatus 400, in a folded configuration 102A. FIG. 30 is a second isometric view illustrating the representative fourth embodiment of a guidewire stabilizing apparatus 400, in the folded configuration 102A. FIG. 31 is a plan view illustrating the representative fourth embodiment of the guidewire stabilizing apparatus 400, in an unfolded, flat configuration 104A. FIG. 32 is a bottom, plan view illustrating the representative fourth embodiment of the guidewire stabilizing apparatus 400, in the folded configuration 102A, with a U-shaped adhesive layer 195A and a U-shaped adhesive cover sheet 198A.

FIG. 33 is an isometric view illustrating a representative embodiment of a kit or set of catheterization instruments 500 having the representative first embodiment of a guidewire stabilizing apparatus 100, in the folded configuration 102.

Referring to FIGS. 1-33, each of the guidewire stabilizing apparatuses 100, 200, 300, 400 share numerous, common features, and function identically to each other. The guidewire stabilizing apparatuses 100, 200, 300, 400 differ from each other insofar as the guidewire stabilizing apparatuses 100, 400 may be constructed by folding (and thereby creating additional structure for) a single, flat sheet 106 of substrate material 108, from the unfolded configurations 104, 104A illustrated in FIG. 2 and FIG. 31, respectively, into the folded configuration 102, 102A, illustrated in FIGS. 1, 3-14, 33, and 29-30, respectively. While the unfolded configuration 104, 104A has all of the structural components of the guidewire stabilizing apparatus 100, 400, because it does not yet have the additional, folded, three-dimensional structure of the guidewire stabilizing apparatus 100, 400, in this unfolded configuration, it is referred to as the guidewire stabilizing apparatus 100A, 400A, respectively. Stated another way, the guidewire stabilizing apparatus 100A, 400A may be considered the precursor form of the guidewire stabilizing apparatus 100, 400, respectively. The guidewire stabilizing apparatus 100, 400 is utilized for guidewire 90 stabilization in the folded configuration 102, 102A. In a representative embodiment, for example and without limitation, the guidewire stabilizing apparatus 100, 400 is typically provided to the end user in this folded configuration 102, 102A, as shown in the kit or set of catheterization instruments 500 illustrated in FIG. 33 for the guidewire stabilizing apparatus 100 (or 400). Alternatively, the guidewire stabilizing apparatus 100, 400 could be provided to the end user in the unfolded configuration 104, 104A, and folded by medical or surgical personnel into the folded configuration 102, 102A for use during the catheterization procedure.

Each of the guidewire stabilizing apparatuses 100, 200, 300, 400 has a respective base 125, 125A, 125B, 125C having a respective first, bottom or lower surface 185, 185A, 185B, 185C, with the respective base 125, 125A, 125C also having a second, upper surface 190, 190A, 190C. The guidewire stabilizing apparatuses 200 and 300 differ from the guidewire stabilizing apparatuses 100, 400 insofar as the guidewire stabilizing apparatuses 200, 300 are not necessarily assembled or manufactured by folding a single, flat sheet 106 of substrate material 108. Accordingly, the guidewire stabilizing apparatuses 200 and 300 do not include the corresponding structures for folding and assembly (e.g., various tabs 140A, 14B, 145A, 145B, though-holes 150A, 150B, 158, and various cut lines 130, 135A, 135B), and other features to enable folding and securing of a sheet 106 of substrate 108 into a three-dimensional guidewire stabilizing apparatuses 100, 400. The guidewire stabilizing apparatuses 200 and 300 may also differ from the guidewire stabilizing apparatuses 100, 400 insofar as the guidewire stabilizing apparatuses 200, 300 may also have differently shaped lateral side walls, for example, and otherwise the guidewire stabilizing apparatuses 200, 300 share many identical structures and have the identical functions of the guidewire stabilizing apparatuses 100 and 400, as mentioned above and as described in greater detail below. The guidewire stabilizing apparatuses 200 and 300 differ from each other insofar as the guidewire stabilizing apparatus 200 includes a larger base 125A compared to the base 125B of the guidewire stabilizing apparatus 300, and otherwise they share identical structures and have the identical functions.

The guidewire stabilizing apparatus 400 differs from the guidewire stabilizing apparatus 100 insofar as: (1) the guidewire stabilizing apparatus 400 has a different, wider configuration or shape for the guidewire stabilization channel 105A as illustrated in FIGS. 29-31, which may be generally described as a more complex shape comprising a square superimposed on a rectangle and further having curvilinear corners or edges, compared to the more rectangular-shaped or configured guidewire stabilization channel 105 of the guidewire stabilizing apparatus 100; (2) the guidewire stabilizing apparatus 400 includes other indicia 85A (textual, “ACCESS”) and 85B (symbolic, arrow) applied to the upper surface 190C of the base 125C (and does not include indicia 80, 85 which may be formed as through-holes in the flat sheet 106 of substrate material 108); and (3) the guidewire stabilizing apparatus 400 utilizes a “U”-shaped adhesive layer 195A and an adhesive cover sheet 198A (which also does not cover the various tabs 140A, 14B, 145A, 145B), and otherwise the guidewire stabilizing apparatus 400 shares the identical structures and has the identical functions as the guidewire stabilizing apparatus 100. Those having skill in the art will also recognize, as illustrated in the first and second isometric views of the guidewire stabilizing apparatus 400 of FIGS. 29 and 30 and in the plan view illustrating the precursor guidewire stabilizing apparatus 400A in the unfolded, flat configuration 104A, that: (1) the first lateral side 160C of the guidewire stabilizing apparatus 400 is identical to the first lateral side 160 of the guidewire stabilizing apparatus 100 (illustrated in FIG. 4); (2) the second lateral side 165C of the guidewire stabilizing apparatus 400 is identical to the second lateral side 165 of the guidewire stabilizing apparatus 100 (illustrated in FIG. 5); (3) the third lateral side 170C of the guidewire stabilizing apparatus 400 is identical to the third lateral side 170 of the guidewire stabilizing apparatus 100 (illustrated in FIG. 6); (4) the fourth lateral side 175C of the guidewire stabilizing apparatus 400 is identical to the fourth lateral side 175 of the guidewire stabilizing apparatus 100 (illustrated in FIG. 7); and (5) the top, upper side 180C of the guidewire stabilizing apparatus 400 differs from the top, upper side 180 of the guidewire stabilizing apparatus 100 solely with respect to the shape or configuration of the corresponding guidewire stabilization channel 105A, 105 of the respective top, upper side 180C, 180. (Accordingly, additional elevational or side views of the guidewire stabilizing apparatus 400 therefore would be redundant with FIGS. 4-7 and are not included or otherwise duplicated herein.)

As a consequence, unless the context so requires or is explicitly indicated to the contrary, any reference to any of the guidewire stabilizing apparatuses 100, 200, 300, 400 and their elements, features, and functions shall be understood to mean and include any of the other guidewire stabilizing apparatuses 100, 200, 300, 400 and their respective elements, features and functions.

Referring to FIGS. 1-14 and 29-31, the guidewire stabilizing apparatus 100A, 400A is typically die cut as a single, flat sheet 106 of substrate material 108 to form various channels tabs, holes, and flaps, as discussed in greater detail below, which is then folded and assembled into the folded configuration 102, 102A of the guidewire stabilizing apparatus 100, 400. As illustrated in FIGS. 2 and 31, scoring cuts 130 and through-cuts (135A, 135B) are utilized, with scoring cuts 130 partially penetrating the substrate material 108 (not cutting entirely through the thickness of the substrate material 108), and the through-cuts 135A, 135B (respectively forming first and second tabs 140A, 140B and extension panel 146) fully penetrating the substrate material 108 (cutting entirely through the thickness of the substrate material 108). Alternatively to scoring cuts 130, embossing may be utilized (to form the fold lines, discussed in greater detail below). In addition, the individual first and second stabilization slits 110A, 115A (of the first pair of stabilization slits 155A) and the individual first and second stabilization slits 110B, 115B (of the second pair of stabilization slits 155B) are formed by corresponding through-cuts. A guidewire stabilization channel 105, 105A (terminating at each end 142, 144 in V-shaped, angled wire guides 120A, 120B), third and fourth tabs 145A, 145B, and first, second, and third tab through-holes 158, 150A, 150B, are also formed by corresponding through-cuts, with the respective first, second, and third tab through-holes 158, 150A, 150B extending through the entire thickness of the single, flat sheet 106 of substrate material 108. Optional indicia 80, 85 may also be formed by corresponding through-cuts (as illustrated in FIGS. 1-14, 33), or by printing or embossing on the flat sheet 106 of substrate material 108, as illustrated in FIGS. 29-31 for text indicia 85A and arrow indicia 85B, for example and without limitation. Optionally, other indicia such as additional text may also be formed on the flat sheet 106 of substrate material 108, such as what gauge of guidewire 90 should be utilized (e.g., 0.014 inch or 0.035 inch). It should also be noted that the single, flat sheet 106 of substrate material 108 is also typically formed by corresponding through-cuts through a larger sheet or roll of substrate material 108, to form the individual guidewire stabilizing apparatus 100A, 400A in the unfolded configuration 104, 104A. Typically, all of these cuts (or embossing) may be made simultaneously using a corresponding die (not separately illustrated).

From the flat, unfolded configuration 104, 104A, the single, flat sheet 106 of substrate material 108 is typically folded along the lines provided by the various scoring cuts 130 (or embossing, not separately illustrated), folding in a direction (into the illustrated plane of the single, flat sheet 106 of substrate material 108) in a way that opens or expands each of the scoring cuts 130, folding at lines 122, 124, 126, 128, 132, 134, 136, and 138 of scoring cuts 130 (or corresponding embossing lines). As the single, flat sheet 106 is folded: (1) first and second tabs 140A, 140B are inserted into the first tab through-hole 158, with the extension panel 146 tucked underneath or below what has now become the fifth, upper side 180, on the second, upper surface 190 of the base, i.e., between the fifth, upper side 180 and the base 125; (2) third tab 145A is inserted into the second tab through-hole 150A; and (3) fourth tab 145B is inserted into the third tab through-hole 150B (as illustrated in FIGS. 8 and 32). The resulting folded configuration 102, 102A of the guidewire stabilizing apparatus 100, 400, respectively, is illustrated in FIGS. 1, 3-14, 33 and 29-30. Optional indicia 85 may be provided, such as to indicate how to remove the guidewire stabilizing apparatus 100, 400 from any associated packaging tray 505 or other packaging (illustrated in FIG. 33), while other optional indicia 80 may be provided, such as to indicate where to insert a guidewire 90. Other or additional (and also optional) indicia 85A, 85B may be provided, such as to indicate how to position the guidewire stabilizing apparatus 100, 400 on the procedure table covering with respect to the catheter access point of the patient or subject (i.e., an orientation of the guidewire stabilizing apparatus with respect to a catheter access point) (illustrated in FIGS. 29 and 30), for example and without limitation.

In addition, to illustrate the structure of the tabs 145A, 145B, 140A, and 140B penetrating through the substrate 108 (forming a base (or base side) 125) to the lower, bottom surface 185 of the guidewire stabilizing apparatus 100, the adhesive layer 195, 195A and the adhesive cover sheet 198, 198A are not illustrated in FIGS. 4-8. Following the folding, an adhesive layer 195, typically supplied with and already having a removable adhesive cover sheet 198, such as a double-sided tape, is applied to the first, lower or bottom surface 185 of the base 125 (which also compresses and flattens the tabs 145A, 145B, 135A, and 135B against the first, lower or bottom surface 185 of the base 125, as illustrated in FIG. 14. Alternatively, a U-shaped adhesive layer 195A, also typically supplied with and already having a removable adhesive cover sheet 198A, such as a double-sided tape, is applied to the first, lower or bottom surface 185, 185C of the base 125, 125C, without also covering any of the various protruding tabs 145A, 145B, 135A, and 135B. More particularly, the adhesive layer 195, 195A has a first adhesive side 162 and a second adhesive side 164, with the first adhesive side 162 of the adhesive layer 195, 195A coupled to the first, lower surface 185, 185C of the base 125, 125C (typically after removing another adhesive cover sheet (not separately illustrated) and adhering the first adhesive side 162 to he base 125, 125C, with the removable adhesive cover sheet 198, 198A being removably coupled to the second adhesive side 164 of the adhesive layer 195, 195A. The guidewire stabilizing apparatus 200, 300 is also typically fabricated to include the adhesive layer 195, 195A having the removable adhesive cover sheet 198, 198A. Any type of suitable adhesive may also be utilized to form the adhesive layer 195, 195A, and any and all materials and ways to adhere or otherwise secure the guidewire stabilizing apparatus 100, 200, 300, 400 to a covering of the operating or procedure table are considered equivalent and within the scope of the disclosure.

In operation, when deployed in a medical, surgical or other interventional catheterization procedure, the removable adhesive cover sheet 198, 198A is peeled away from the second adhesive side 164 of the adhesive layer 195, 195A and the exposed second adhesive side 164 of the adhesive layer 195, 195A (coupled to the first, lower or bottom surface 185, 185A, 185B, 185C of the base 125, 125A, 125B, 125C) is then pressed against and adhered to the sterile covering of the operating or procedure table, thereby stably attaching the guidewire stabilizing apparatus 100, 200, 300, 400 to the sterile covering of the operating or procedure table. A sufficiently strong adhesive layer 195, 195A is utilized to stably hold the guidewire stabilizing apparatus 100, 200, 300, 400 against the operating or procedure table throughout the medical, surgical or other catheterization interventional procedure. Following the medical, surgical or other interventional catheterization procedure, the guidewire stabilizing apparatus 100, 200, 300, 400 is typically removed along with the other covering materials, sterile drapes, and other single-use instruments, and disposed of properly.

In the folded configuration 102, 102A, the guidewire stabilizing apparatus 100, 400 comprises a base 125, 125C (formed by the substrate 108) supporting four sides, a first lateral side 160, 160C and a second lateral side 165, 165C (opposite the first lateral side 160, 160C respectively), and a third lateral side 170, 170C and a fourth lateral side 175, 175C (opposite the third lateral side 170, 170C, respectively), which are integrally formed with a top, upper side 180, 180C. With the four lateral sides 160 (160C), 165 (165C), 170 (170C), 175 (175C), the top, upper side 180 (180C) is a fifth, upper side 180 (180C) and the base 125 (125C) is a sixth, lower side, collectively forming a box structure, as illustrated. The guidewire stabilizing apparatus 200 also comprises a base 125A supporting four lateral sides, a first lateral side 160A and a second lateral side 165A (opposite the first lateral side 160A), and a third lateral side 170A and a fourth lateral side 175A (opposite the third lateral side 170A), which are coupled to or integrally formed with a top, upper side 180A. The guidewire stabilizing apparatus 300 also comprises a comparatively smaller base 125B supporting four sides, a first lateral side 160B and a second lateral side 165B (opposite the first lateral side 160B), and a third lateral side 170B and a fourth lateral side 175B (opposite the third lateral side 170B), which are coupled to or integrally formed with a top, upper side 180B.

When folded, it can be seen that: (1) the first and second tabs 140A, 140B and the extension panel 146 are integrally formed with or otherwise coupled to or part of the first lateral side 160, 160C, with the first and second tabs 140A, 140B extending through the base 125, 125C to be underneath the base 125, 125C along the lower, bottom surface 185, 185C of the base 125, 125C, and with the extension panel 146 tucked underneath or below what has now become the fifth, upper side 180, 180C, between the fifth, upper side 180, 180C and the base 125, 125C; (2) the third tab 145A is integrally formed with or otherwise coupled to or part of the third lateral side 170, 170C, with the third tab 145A extending through the base 125, 125C to be underneath the base 125, 125C along the lower, bottom surface 185, 185C of the base 125, 125C; and (3) the tab 145B is integrally formed with or otherwise coupled to or part of the fourth lateral side 175, 175C, with the tab 145B extending through the base 125, 125C to be underneath the base 125, 125C along the lower, bottom surface 185, 185C of the base 125, 125C.

Each of the guidewire stabilizing apparatuses 100, 200, 300, 400 further comprises a guidewire stabilization channel 105, 105A extending across the top, upper side 180, 180A, 180B, 180C and also extending down into the first lateral side 160, 160A, 160B, 160C and the second lateral side 165, 165A, 165B, 165C. More particularly, the guidewire stabilization channel 105, 105A is formed in the top, upper side 180, 180A, 180B, 180C of the corresponding guidewire stabilizing apparatuses 100, 200, 300, 400. The guidewire stabilization channel 105, 105A further extends into and terminates (at a first end 142) in a first V-shaped angled wire guide 120A extending in the first lateral side 160, 160A, 160B, 160C and (at a second end 144) in a second V-shaped angled wire guide 120B extending in the second lateral side 165, 165A, 165B, 165C, as illustrated. At the pointed termination or end 142, 144 of each “V”, also as illustrated, a corresponding vertical, first stabilization slit 110A, 110B is provided in each corresponding first lateral side 160, 160A, 160B, 160C and second lateral side 165, 165A, 165B, 165C, respectively, and a corresponding horizontal, second stabilization slit 115A, 115B is provided in each first lateral side 160, 160A, 160B, 160C and second lateral side 165, 165A, 165B, 165C, respectively. Each second stabilization slit 115A, 115B crosses the respective first stabilization slit 110A, 110B, generally at a right (90°) angle, and generally at one-half (½) to about two-thirds (⅔) of the vertical length of the first stabilization slit 110A, 110B measured or determined from the pointed termination or end 142, 144 of the V-shaped angled wire guide 120A, 120B, as illustrated.

In various representative embodiments, depending upon the selected type and properties of substrate material 108, the thickness of the flat sheet 106 of the selected substrate material 108, and the diameter of the guidewire 90, each first stabilization slit 110A, 110B and each second stabilization slit 115A, 115B has a length generally between 1/16^{th} to 3/16^{th} of an inch (1.5875 to 4.7625 mm). More particularly, in various representative embodiments, each first stabilization slit 110A, 110B and each second stabilization slit 115A, 115B has a length generally one-eighths (⅛) of an inch (3.175 mm) for the type and thickness of the selected substrate material 108 and for a 0.035 inch (0.889 mm) or 0.014 inch (0.3556 mm) diameter guidewire 90. In general, the size (length) of each of the first and second stabilization slits 110A, 110B, 115A, 115B may be selected based upon the size, gauge, flexibility/stiffness, springiness, and type of guidewire 90 being utilized and the type and thickness of the substrate material 108 being utilized. An addition, also within the scope of the disclosure, in other representative embodiments, each of the first stabilization slit 110A, 110B and the second stabilization slit 115A, 115B may have differing lengths.

The first stabilization slit 110A and second stabilization slit 115A collectively form a first pair of stabilization slits 155A (in the first lateral side 160, 160A, 160B, 160C) having a plurality of flexible, deformable stabilization flaps, a first deformable flap 112A, a second deformable flap 114A, a third deformable flap 116A, and a fourth deformable flap 118A. Also, the first stabilization slit 110B and second stabilization slit 115B collectively form a second pair of stabilization slits 155B (in the second lateral side 165, 165A, 165B, 165C) having a plurality of flexible, deformable stabilization flaps, a first deformable flap 112B, a second deformable flap 114B, a third deformable flap 116B, and a fourth deformable flap 118B. In addition, on each side of the first (vertical) stabilization slit 110A, 110B, the respective lateral side 160, 165 is also flexible, deformable, deflectable, or displaceable.

During a medical, surgical or other interventional catheterization procedure, following insertion of a distal end of a guidewire 90 into the patient or subject and movement of the distal end of the guidewire 90 to a desired or selected location within the patient or subject, the guidewire 90 may then be stabilized by using a guidewire stabilizing apparatus 100, 200, 300, 400. Generally, one guidewire stabilizing apparatus 100, 200, 300, 400 is sufficient to stabilize a guidewire 90, although additional guidewire stabilizing apparatuses 100, 200, 300, 400 may also be utilized, as an option. The adhesive cover sheet 198, 198A is removed from the adhesive layer 195, 195A of a guidewire stabilizing apparatus 100, 200, 300, 400, and the base 125, 125A, 125B, 125C having the exposed adhesive layer 195, 195A of the guidewire stabilizing apparatus 100, 200, 300, 400 is placed by the user (such as medical personnel) at a desired or selected location along the operating or procedure table, and pressed into any covering over the operating or procedure table at this desired or selected location. The user (such as medical personnel) typically applies pressure to the guidewire stabilizing apparatuses 100, 200, 300, 400 to stably adhere the guidewire stabilizing apparatus 100, 200, 300, 400 to the covering over the operating or procedure table.

Following secure placement of the guidewire stabilizing apparatus 100, 200, 300, 400 at the desired or selected location, a proximal portion of the guidewire 90 is held taught by the user (such as medical personnel), placed into the stabilization channel 105, 105A and into the V-shaped angled wire guides 120A, 120B, and then pushed or snapped down into each of the respective first pair of stabilization slits 155A and second pair of stabilization slits 155B. More specifically, the proximal portion of the guidewire 90 is pushed or snapped into the respective first stabilization slit 110A of the first lateral side 160, 160A, 160B, 160C and into the first stabilization slit 110B of the second lateral side 165, 165A, 165B, 165C, and generally also into the respective second stabilization slit 115A of the first lateral side 160, 160A, 160B, 160C and the second stabilization slit 115B of the second lateral side 165, 165A, 165B, 165C, with the guidewire 90 typically catching and stopping at about or in the vicinity of the intersection of the first stabilization slit 110A, 110B with the second stabilization slit 115A, 115B of the respective first lateral side 160, 160A, 160B, 160C and second lateral side 165, 165A, 165B, 165C, as illustrated in FIGS. 10-14. The guidewire 90 is thereby stabilized (and held under tension) at a plurality of separate locations, namely, at least two (or more) in-line stabilization points created by: (1) the first pair of stabilization slits 155A and/or the first stabilization slit 110A; and (2) the second pair of stabilization slits 155B and/or the first stabilization slit 110B, thereby holding a single wire in multiple stabilization slits providing corresponding multiple points of stabilizing contact.

While potentially not quite as robust, those having skill in the art will recognize that guidewire 90 stabilization may be provided using one or more simpler versions of a guidewire stabilizing apparatus 100, 200, 300, 400, such as one having fewer than four lateral sides 160-160C, 165-165C, 170-170C, 175-175C, for example and without limitation. Also for example and without limitation, not separately illustrated, a guidewire stabilizing apparatus may be formed having a triangular box shape having three lateral sides, with a stabilization channel 105, 105A also extending across an upper side 180 between two adjacent lateral sides of the triangle, and terminating in corresponding angled wire guides 120A, 120B with one or more corresponding first stabilization slits 110A, 110B and/or a pair of stabilization slits 155A, 155B.

The guidewire 90 is held in place in each pair of pair of stabilization slits 155A, 155B and/or each of the first stabilization slits 110A, 110B, generally through the force (pressure and friction) exerted by the deformation or deflection of the respective pair of stabilization slits 155A, 155B, and/or each side of the vertical, first stabilization slits 110A, 110B of the respective first and second lateral sides 160-160C, 165-165C, and/or various combinations of the deformable flaps 112A, 114A, 116A, 118A and the deformable flaps 112B, 114B, 116B, 118B of the respective first and second lateral sides 160-160C, 165-165C. The substrate material 108 is generally selected to have sufficient strength and flexibility to provide the desired level of force (pressure and friction) sufficient to secure a selected guidewire 90 within each pair of pair of stabilization slits 155A, 155B (and/or within each of the first stabilization slits 110A, 110B and/or various combinations of the deformable flaps 112A, 114A, 116A, 118A and the deformable flaps 112B, 114B, 116B, 118B of the respective first and second lateral sides 160-160C, 165-165C), and may vary depending upon the size, gauge, flexibility/stiffness, springiness, and type of guidewire 90 being utilized. In a representative embodiment, the substrate material 108 may be a plastic such as polyethylene, polycarbonate, polypropylene, polyethylene terephthalate, or high-density polyethylene (“HDPE”, typically having a molecular weight between 100,000-250,000 Daltons), for example and without limitation, and other suitable materials are also discussed in greater detail below, any and all of which are considered equivalent and within the scope of the disclosure. In a representative embodiment, the substrate material 108 has a thickness ranging from 0.020 to 0.035 inches (0.508 to 0.889 mm), also for example and without limitation, with larger or small thicknesses also considered equivalent and within the scope of the disclosure. Also in a representative embodiment, the guidewire stabilizing apparatus 100, 200, 300, 400 is designed to provide stability through an applied force (walking or movement force) from the guidewire 90 of at least up to and possibly exceeding 0.50 lbf (2.224 N), for example and without limitation.

As mentioned above, the guidewire 90 may be held in place in several different ways by the guidewire stabilizing apparatus 100, 200, 300, 400, such as in the first arrangement 92 for the guidewire 90 illustrated in FIG. 11 or the second arrangement 94 for the guidewire 90 illustrated in FIG. 12. The guidewire 90 may be held in place through the deformation of one or more combinations of one or more of the plurality of flexible, deformable stabilization flaps, namely, one or more combinations of the first, second, third, and fourth deformable flaps 112A, 114A, 116A, 118A of the first lateral side 160, 160A, 160B, 160C and the first, second, third, and fourth deformable flaps 112B, 114B, 116B, 118B of the second lateral side 165, 165A, 165B, 165C. As illustrated in FIG. 11, following insertion of the guidewire 90, the various deformable flaps 112A, 114A, 116A, and 118A have become curved and deformed, pressing against and securing the guidewire 90 within the first and second stabilization slits 110A, 115A. As illustrated in FIG. 12, following insertion of the guidewire 90, in addition to any deformation of the various deformable flaps 112A, 114A, 116A, and 118A, each lateral side of the vertical, first stabilization slit 110A has been displaced, such that the respective portions of the first lateral side 160, 160A, 160B, 160C adjacent or lateral to the first stabilization slit 110A has been flexed, deflected, or displaced, also pressing against and securing the guidewire 90 within the first stabilization slits 110A and within at least a portion of the second stabilization slit 115A. It should be noted that in operation during a medical, surgical, or interventional catheterization procedure, any number of various combinations of deformation of one or more of the deformable flaps 112A, 114A, 116A, 118A of the first lateral side 160, 160A, 160B, 160C and deformable flaps 112B, 114B, 116B, 118B of the second lateral side 165, 165A, 165B, 165C may occur, and any and all such variations are considered equivalent and within the scope of the disclosure.

It should also be noted that while the guidewire 90 is illustrated in FIGS. 10-14 as substantially straight and parallel with the lateral sides of the stabilization channel 105, those having skill in the art will recognize that the guidewire 90 may also have some curve or curvature within the stabilization channel 105, 105A, depending upon the angle of the guidewire 90 as it is being held in each pair of stabilization slits 155A, 155B. Any and all such variations are expected, considered equivalent, and within the scope of this disclosure.

Another significant feature of the guidewire stabilizing apparatus 100, 200, 300, 400 is that the stabilized guidewire 90, while in place within each pair of stabilization slits 155A, 155B, may nonetheless be manually slid by the user (medical personnel) through each pair of stabilization slits 155A, 155B, without having to clamp and unclamp the guidewire 90 or remove the guidewire 90 from the pairs of stabilization slits 155A, 155B.

As mentioned above, the guidewire stabilizing apparatus 100, 200, 300, 400 are capable of being integrated and included into the overall, sterile packaging of the catheterization instruments, to be provided as part of a single, complete kit or set of instruments 500 for the medical, surgical or other interventional catheterization procedure. Referring to FIG. 33, such a representative kit or set of instruments 500 may comprise a guidewire stabilizing apparatus 100, 200, 300, 400, a catheter 515 (which typically also comprises a flush valve 525), a syringe 520, and various other components such as additional catheter tubing, all arranged and spaced apart on an associated packaging tray 505 or other sterile packaging. While illustrated in FIG. 33 with guidewire stabilizing apparatus 100, those having skill in the art will recognize that any guidewire stabilizing apparatus 100, 200, 300, 400 may be included equivalently within a kit or set of instruments 500, and any and all such variations are within the scope of the disclosure.

Continuing to refer to FIG. 33, the packaging tray 505 has a plurality of tabs 510 or other packaging tray extensions 530 configured to hold or secure each of the guidewire stabilizing apparatus 100 (or 200, 300, 400), the catheter 515, and the syringe 520, along with other components such as the flush valve 525, for example and without limitation. As illustrated, the guidewire stabilizing apparatus 100 (or, equivalently, any other the guidewire stabilizing apparatus 200, 300, 400) is coupled to or otherwise held or secured on the associated tray 505 or other packaging by a plurality of tabs 510 formed within the associated tray 505 or other packaging, and the guidewire stabilizing apparatus 100 may be removed from the associated tray 505 or other packaging by sliding or pulling the guidewire stabilizing apparatus 100 out of or away from the tabs 510. The various other components of the kit or set of instruments 500 are also held in place or otherwise secured on the associated tray 505 or other packaging by packaging tray extensions 530 (or other holding tabs), as illustrated. Not separately illustrated in FIG. 33, the kit or set of instruments 500 may also be covered with a sterile covering, such as a sterile shrink wrap. While the kit or set of instruments 500 illustrated in FIG. 33 is shown as including a guidewire stabilizing apparatus 100, those having skill in the art will recognize that the kit or set of instruments 500 may include any embodiment of the guidewire stabilizing apparatus 100, 200, 300, 400, and any combination of instruments such as catheters 515, syringes 520, etc., and any and all such variations are considered equivalent and within the scope of the disclosure.

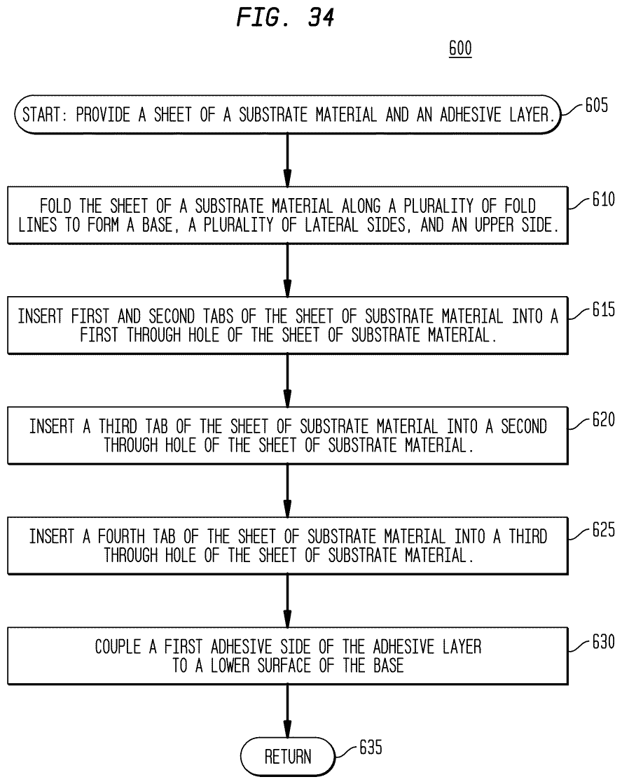

FIG. 34 is a flow chart illustrating a representative embodiment for a method 600 to manufacture the representative first and fourth embodiments of the guidewire stabilizing apparatus 100, 400, and provides a useful summary. The method 600 begins, start step 605, with providing a sheet 106 of a substrate material 108 and an adhesive layer 195, 195A. The sheet 106 of a substrate material 108 has or has had a plurality of through-cuts, the plurality of through-cuts forming the first, second, third, and fourth tabs 135A, 135B, 145A, 145B and forming the first, second, and third through-holes 158, 150A, 150B, with the sheet 106 of a substrate material 108 further having a plurality of fold lines 122, 124, 126, 128, 132, 134, 136, 138, such as formed from embossing or scoring cuts 130. As mentioned above, the fabrication of this sheet 106 of substrate material 108 may be provided by die cutting, and may be provided, for example, by purchasing the cut sheet of substrate material 108 from a third-party fabricator. In addition, the adhesive layer 195, 195A is typically a pre-fabricated product which also may be purchased from a third-party, such as a double-sided adhesive tape, with the adhesive layer 195, 195A having a first adhesive side 162 and a removable adhesive cover sheet 198, 198A on a second adhesive side 164 of the adhesive layer 195, 195A.

Referring to FIG. 34, the sheet 106 of a substrate material 108 is then folded along the plurality of fold lines, step 610, to form the base 125 and a plurality of lateral sides 160, 165, 170, 175. The first and second tabs 135A, 135B of the sheet 106 of the substrate material 108 are inserted into the first through-hole 158 of the sheet 106 of the substrate material 108, step 615. The third tab 145A of the sheet 106 of the substrate material 108 is inserted into the second through-hole 150A of the sheet 106 of the substrate material 108, step 620, and the fourth tab 145B of the sheet 106 of the substrate material 108 is inserted into the third through-hole 150B of the sheet 106 of the substrate material 108, step 625. In step 630, a first adhesive side 162 of an adhesive layer 195, 195A is coupled to the first, lower surface 185, 185C of the base 125, 125C, with the adhesive layer 195, 195A having a removable adhesive cover sheet 198, 198A on a second adhesive side 164 of the adhesive layer 195, 195A. Following step 630, the manufacturing (or fabrication) method may end, return step 635.

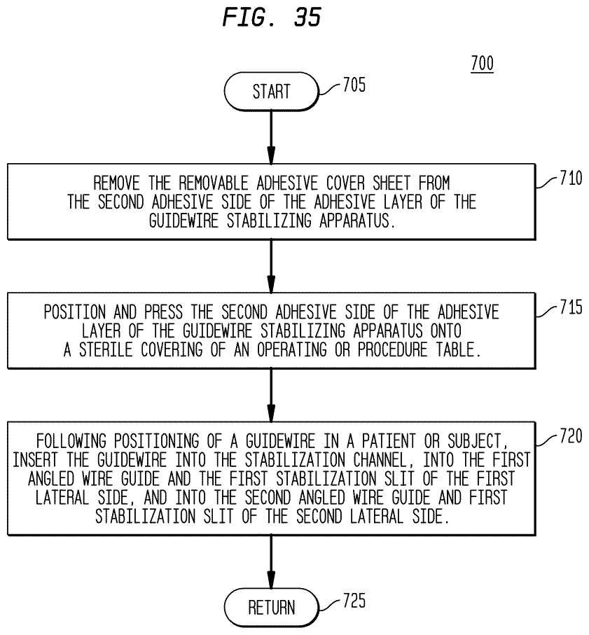

FIG. 35 is a flow chart illustrating a representative embodiment for a method 700 to use the various embodiments of the guidewire stabilizing apparatus 100, 200, 300, 400 in a catheterization procedure, and also provides a useful summary. The method begins, start step 705, with removing the removable adhesive cover sheet 198, 198A from the second adhesive side 164 of the adhesive layer 195, 195A of the guidewire stabilizing apparatus 100, 200, 300, 400, step 710. The second adhesive side 164 of the adhesive layer 195, 195A of the guidewire stabilizing apparatus 100, 200, 300, 400 is then positioned over and pressed onto a sterile covering of an operating or procedure table, step 715. Following positioning of a guidewire in a patient or subject, the guidewire is inserted into the stabilization channel 105, 105A, into the first angled wire guide 120A and the first stabilization slit 110A of the first lateral side 160, 160A, 160B, 160C, and into the second angled wire guide 120B and first stabilization slit 110B of the second lateral side 165, 165A, 165B, 165C, step 720. Following step 720, the method may end, return step 725.

As mentioned above, the guidewire stabilizing apparatus 100, 200, 300, 400 may be fabricated in a wide variety of ways, including by forming from a single sheet 106 of substrate material 108, integrally formed (e.g., injection molded, 3D printed) or assembled from separate components (e.g., using any suitable fasteners or adhesives, not separately illustrated), for example and without limitation, and all such variations are considered equivalent and within the scope of the disclosure. The guidewire stabilizing apparatus 100, 200, 300, 400 may be implemented using any suitable material, and may be opaque or transparent, with suitable materials including any rigid (or semi-flexible) polymer or plastic, such as polyvinylchloride (PVC), polystyrene, polyacrylate, polytetrafluoroethylene (PTFE or Teflon), nylon, polycarbonates, polyesters, carbon fiber, silicone, silicone rubber, a metal, an alloy, etc., for example and without limitation, and all such variations are considered equivalent and within the scope of the disclosure. The guidewire stabilizing apparatus 100, 200, 300, 400 also may have one or more coatings (not separately illustrated), such as an antibiotic or antimicrobial coating, also for example and without limitation.

Representative examples of suitable polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D, L-lactic acid); poly(D, L-lactide); poly(glycolic acid-cotrimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof.

The polymers may further include, but are not limited to, poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical or structural coupling, connection or attachment, or adaptation or capability for such a direct or indirect electrical or structural coupling, connection or attachment, including integrally formed components and components which are coupled via or through another component.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

1. A guidewire stabilizing apparatus, comprising:
a base;
a plurality of lateral sides coupled to or integrally formed with the base, the plurality of lateral sides comprising a first lateral side, a second lateral side opposite the first lateral side, a third lateral side, and a fourth lateral side opposite the third lateral side, the first lateral side comprising a first angled wire guide and the second lateral side comprising a second angled wire guide; and
a fifth, upper side coupled to or integrally formed with the first, second, third, and fourth lateral sides, the fifth, upper side comprising: a stabilization channel extending across the fifth, upper side and terminating in the first angled wire guide in the first lateral side and in the second angled wire guide in the second lateral side;
wherein the first and second lateral sides each further comprise:
a first stabilization slit extending toward the base from the respective first and second angled wire guides; and
a second stabilization slit extending across the first stabilization slit to form a plurality of deformable flaps in each of the respective first and second lateral sides.

2. The guidewire stabilizing apparatus of claim 1, wherein the second stabilization slit is arranged at a right angle to the first stabilization slit.

3. The guidewire stabilizing apparatus of claim 1, wherein the base further comprises a plurality of through-holes, and wherein the first lateral side further comprises:
a first tab; and
a second tab;
wherein the first and second tabs each extend through a first through-hole, of the plurality of through-holes of the base, the first and second tabs further arranged along a first, lower surface of the base.

4. The guidewire stabilizing apparatus of claim 3, wherein the first lateral side further comprises:
an extension panel arranged on a second, upper surface of the base and extending between the fifth, upper side and the second, upper surface of the base.

5. The guidewire stabilizing apparatus of claim 3, wherein the third lateral side further comprises:
a third tab extending through a second through-hole, of the plurality of through-holes of the base, the third tab further arranged along the first, lower surface of the base.

6. The guidewire stabilizing apparatus of claim 5, wherein the fourth lateral side further comprises:
a fourth tab extending through a third through-hole, of the plurality of through-holes of the base, the fourth tab further arranged along the first, lower surface of the base.

7. A method of manufacturing the guidewire stabilizing apparatus of claim 6, from a sheet of a substrate material having a plurality of through-cuts, the plurality of through-cuts forming the first, second, third, and fourth tabs and the first, second, and third through-holes, the sheet of a substrate material further having a plurality of fold lines, the method comprising:
folding the sheet of a substrate material along the plurality of fold lines;
inserting the first and second tabs into the first through-hole;
inserting the third tab into the second through-hole; and
inserting the fourth tab into the third through-hole.

8. The method of claim 7, wherein an adhesive layer comprises a first adhesive side, a second adhesive side, and a removable adhesive cover sheet on the second adhesive side, the method further comprising:
coupling the first adhesive side of the adhesive layer to the first, lower surface of the base.

9. The guidewire stabilizing apparatus of claim 6, wherein the first and second tabs are integrally formed with the first lateral side, the third tab is integrally formed with the third lateral side, and the fourth tab is integrally formed with the fourth lateral side.

10. The guidewire stabilizing apparatus of claim 1, further comprising:
an adhesive layer having a first adhesive side and a second adhesive side, the first adhesive side of the adhesive layer coupled to a first, lower surface of the base; and
a removable adhesive cover sheet coupled to the second adhesive side of the adhesive layer.

11. A method of using the guidewire stabilizing apparatus of claim 10, comprising:
removing the removable adhesive cover sheet;
positioning and pressing the second adhesive side of the adhesive layer to a sterile covering of an operating or procedure table; and
inserting a guidewire into the stabilization channel, into the first angled wire guide and the first stabilization slit of the first lateral side, and into the second angled wire guide and first stabilization slit of the second lateral side.

12. The guidewire stabilizing apparatus of claim 1, wherein the base further comprises:
one or more indicia or indicators, the one or more indicia or indicators configured to indicate at least one indication selected from the group consisting of: an orientation of the guidewire stabilizing apparatus with respect to a catheter access point, a removal from packaging, a location for insertion of a guidewire into the stabilization channel, the first or second angled wire guides, and one or more combinations thereof.

13. (canceled)

14. The guidewire stabilizing apparatus of claim 1, wherein the first stabilization slit and the second stabilization slit each have a length between 1/16th to 3/16th of an inch (1.5875 to 4.7625 mm).

15. The guidewire stabilizing apparatus of claim 1, wherein each of the base, the first lateral side, the second lateral side, the third lateral side, the fourth lateral side, and the fifth, upper side each have a thickness ranging from 0.020 to 0.035 inches (0.508 to 0.889 mm), and wherein the first stabilization slit and second stabilization slit are configured to secure a guidewire having a diameter at or between 0.014 inch (0.3556 mm) to 0.035 inch (0.889 mm) against an applied force of 0.50 lbf (2.224 N).

16. (canceled)

17. The guidewire stabilizing apparatus of claim 1, wherein the guidewire stabilizing apparatus is foldable from a single sheet of substrate material.

18. The guidewire stabilizing apparatus of claim 1, wherein each of the first angled wire guide and the second angled wire guide are V-shaped.

19. The guidewire stabilizing apparatus of claim 1, wherein the third lateral side and the fourth lateral side are each coupled between the first and second lateral sides.

20. The guidewire stabilizing apparatus of claim 1, further comprising a kit of catheterization instruments, the kit of catheterization instruments comprising:
a catheter;
a syringe; and
a packaging tray having a plurality of tabs or other packaging tray extensions configured to hold or secure each of the guidewire stabilizing apparatus, the catheter, and the syringe.

21-26. (canceled)

27. A guidewire stabilizing apparatus, comprising: a base;
a plurality of lateral sides coupled to or integrally formed with the base, a first lateral side of the plurality of lateral sides comprising a first angled wire guide and a second lateral side of the plurality of lateral sides comprising a second angled wire guide; and
an upper side coupled to or integrally formed with the plurality of lateral sides, the upper side comprising: a stabilization channel extending across the upper side and terminating in the first angled wire guide in the first lateral side and in the second angled wire guide in the second lateral side;
wherein the first and second lateral sides each further comprise:
a first stabilization slit extending toward the base from the respective first and second angled wire guides; and
a second stabilization slit extending across the first stabilization slit and at a right angle to the first stabilization slit to form a plurality of deformable flaps in each of the respective first and second lateral sides.

28-30. (canceled)

31. A guidewire stabilizing apparatus, comprising:
a base, the base having a plurality of through-holes;
a plurality of lateral sides coupled to or integrally formed with the base, the plurality of lateral sides comprising a first lateral side, a second lateral side opposite the first lateral side, a third lateral side, and a fourth lateral side opposite the third lateral side, the first lateral side comprising a first angled wire guide and the second lateral side comprising a second angled wire guide, and the first, third, and fourth lateral sides each having one or more tabs coupled in a corresponding through-hole of the plurality of through-holes of the base;
a fifth, upper side coupled to or integrally formed with the first, second, third, and fourth lateral sides, the fifth, upper side comprising: a stabilization channel extending across the fifth, upper side and terminating in the first angled wire guide in the first lateral side and in the second angled wire guide in the second lateral side;
an adhesive layer having a first adhesive side and a second adhesive side, the first adhesive side of the adhesive layer coupled to a lower surface of the base; and
a removable adhesive cover sheet coupled to the second adhesive side of the adhesive layer;
wherein the first and second lateral sides each further comprise:
a first stabilization slit extending toward the base from the respective first and second angled wire guides; and
a second stabilization slit extending across the first stabilization slit and at a right angle to the first stabilization slit to form a plurality of deformable flaps in each of the respective first and second lateral sides.
